(19) United States
(12) Patent Application Publication
(10) Pub. No.: US 2020/0118667 A1
(43) Pub. Date: Apr. 16, 2020

(54) Secure System and Method To Produce Customized Nutritional Products

(71) Applicants: Terry LaCore, Melissa, TX (US); Michael Smith, Melissa, TX (US)

(72) Inventors: Terry LaCore, Melissa, TX (US); Michael Smith, Melissa, TX (US)

(21) Appl. No.: 16/599,084

(22) Filed: Oct. 10, 2019

Related U.S. Application Data
(60) Provisional application No. 62/744,077, filed on Oct. 10, 2018.

(51) Int. Cl. G16H 20/60; G06Q 10/08; G16B 20/20

(52) U.S. Cl. CPC G16H 20/60; G16B 20/20; G06Q 10/083

(57) ABSTRACT

In various implementations, the system may facilitate the customization of nutritional products. The system may track sample(s) and allow testing of sample(s) in compliance with government and/or industry requirements (e.g., laws, regulations, and/or standards). The system may determine a core formulation based on DNA testing of a sample. The system may utilize the core formulation to create customized nutritional products. The system may monitor samples, testing, and/or orders (e.g., to inhibit unauthorized access to user information and/or to provide updates which may increase user satisfaction).

### CROSS-REFRENCE TO RELATED APPLICATION(S)

This application claims the benefit of priority to U.S. Provisional Patent Application 62/744,077 entitled “SYSTEMS AND METHODS TO PRODUCE CUSTOMIZED NUTRITIONAL PRODUCTS” and filed on Oct. 10, 2019, which is hereby incorporated by reference for all purposes.

### TECHNICAL FIELD

The present invention relates to producing nutritional products and a secure network for managing production of customized nutritional products.

### BACKGROUND

In various implementations, customers visit stores and select nutritional supplements. Customers select supplements based on advice from sales agents, packaging, and/or personal preferences. Advice from sales agents can be incomplete and/or uninformed. Users may not be familiar with the different supplements available. Additionally, each user may have underlying conditions that impact the ability of the supplement to help and/or may be impacted by the supplement.

### SUMMARY

In various implementations, the system may obtain user information, monitor user information, identify supplement(s) for the user based on the user information, and deliver the identified supplement(s). The system may be capable of restricting access to at least a portion of the user information to for example, comply with government and/or industry regulations (e.g., privacy laws). The system may communicate with one or more modules of the system and/or third party software to monitor various processes of the system.

The details of one or more implementations are set forth in the accompanying drawings and the description below. Other features, objects, and advantages of the implementations will be apparent from the description and drawings.

### BRIEF DESCRIPTION OF THE DRAWINGS

For a more complete understanding of this disclosure and its features, reference is now made to the following description, taken in conjunction with the accompanying drawings, in which:

FIG. 1 illustrates an implementation of an example process for producing customized nutritional products. FIGS. 1A-1F illustrate portions of the example process illustrated in FIG. 1.

FIG. 2 illustrates an implementation of an example system that may be utilized to perform one or more operations, such as the operation(s) illustrated in FIG. 1.

Like reference symbols in the various drawings indicate like elements.

### DETAILED DESCRIPTION

Nutritional products may improve and/or maintain the health of a user (e.g., sick and/or healthy individuals). Nutritional products may include daily supplements and/or other types of supplements (e.g., boosts, cleanses, etc.). Nutritional products may come in various administration forms such as drinks (e.g., shakes, teas, and/or other types of drinks), foods, capsules, tablets, powders, additives (e.g., to be added to foods or drinks), etc.

In various implementations, the system may help users identify and/or select nutritional products based on user information. The nutritional products may include a first portion customized for the user (e.g., core formulation), a second portion standardized for a user (e.g., a pre-nutrition), and/or third portion(s) associated with the nutritional product (e.g., tea, shake, capsule, sweetener, etc.). The portion customized for the user may be based on analysis of the DNA or a portion thereof of a user.

In various implementations, the system may obtain information related to a DNA sample (e.g., DNA or a portion thereof) of the user and determine a core formulation (e.g., for creation of a customized portion of a nutritional product) based on the information related to the DNA sample. The system may allow creation of customized nutritional product(s) based on the core formulation and may allow delivery of the customized nutritional product(s). The system may inhibit access to information related to a DNA sample, which may increase user satisfaction (e.g., since users may feel more comfortable with providing DNA information) and/or may increase compliance with government and/or industry regulations (e.g., privacy laws and/or regulations such as Health Insurance Portability and Accountability Act “HIPAA”). A user may be administered the customized nutritional product(s) to improve and/or maintain the health of the individual. Since the nutritional product(s) may be customized based on information related to the user's DNA, the nutritional product(s) may be more effective at improving and/or maintaining the health of the individual than nutritional product(s) purchased off the shelf, for example.

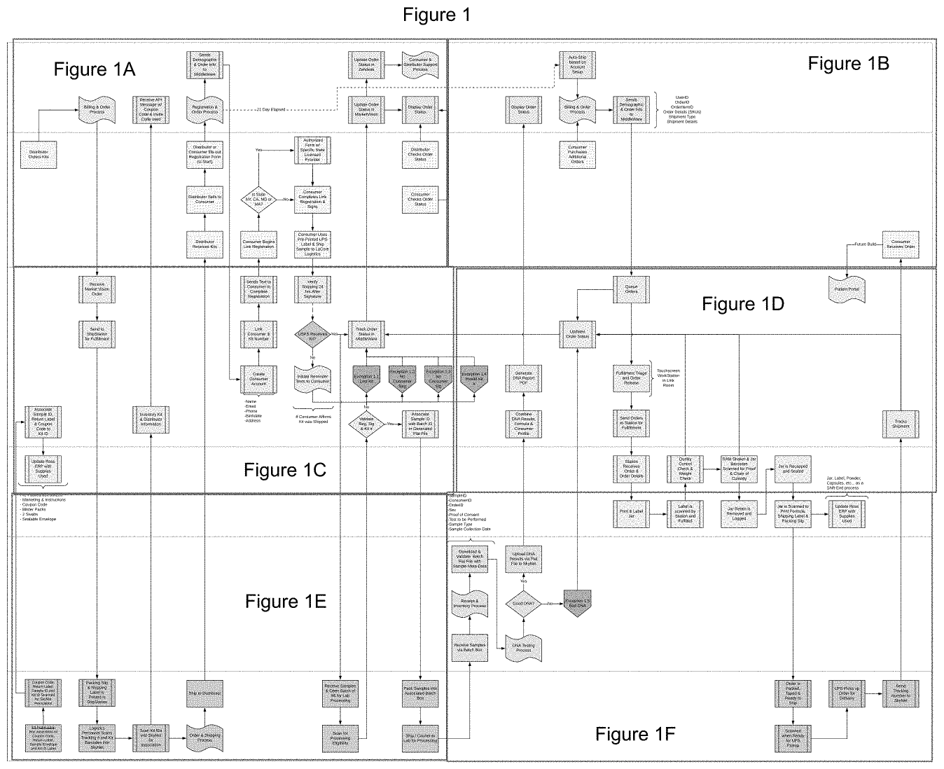

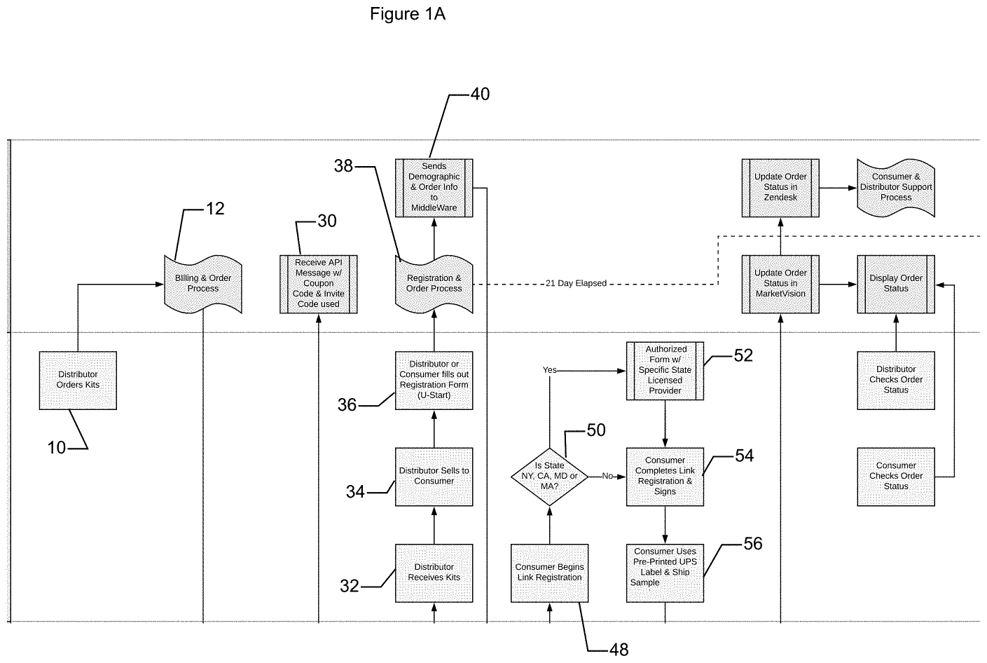

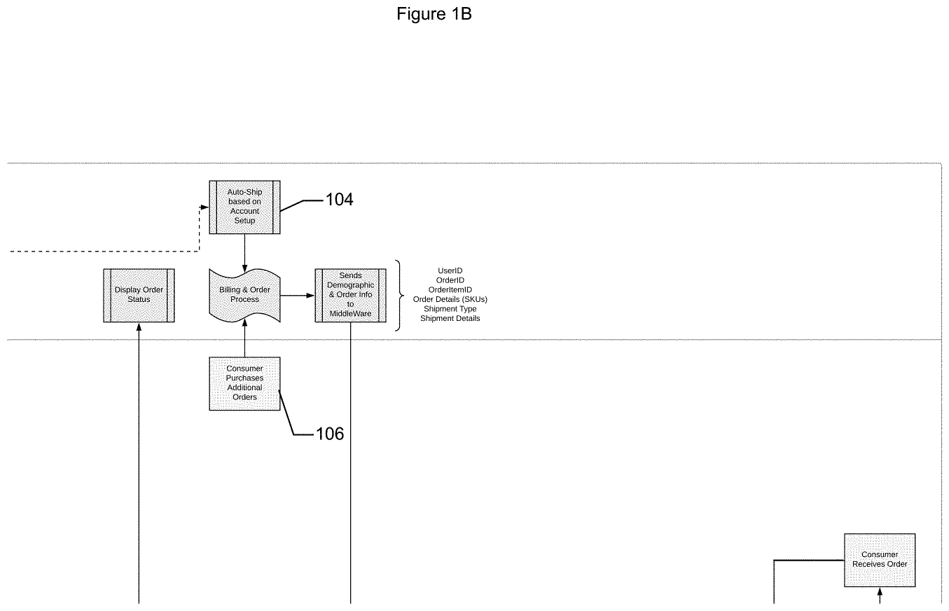

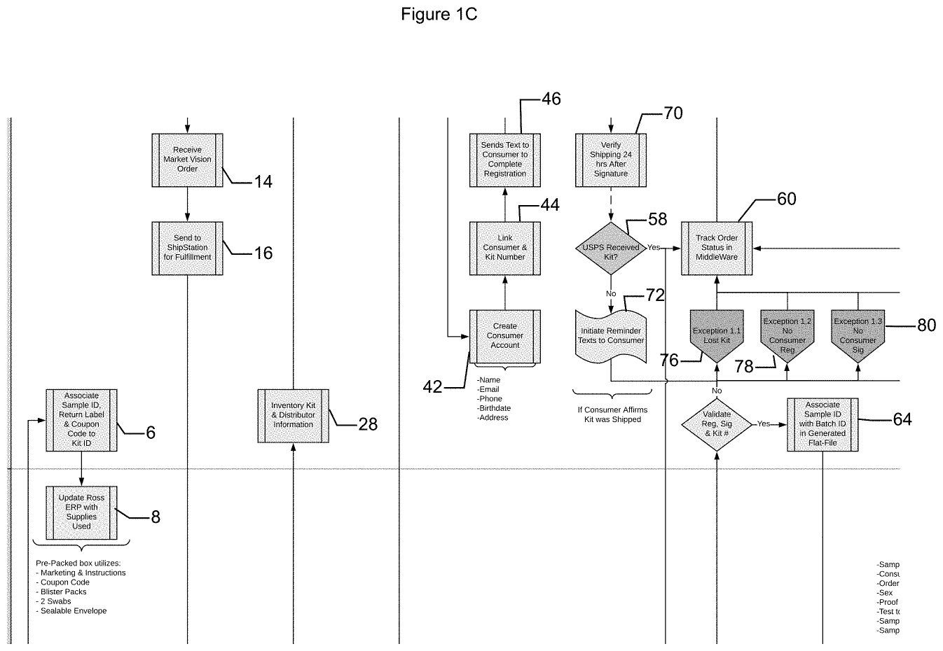

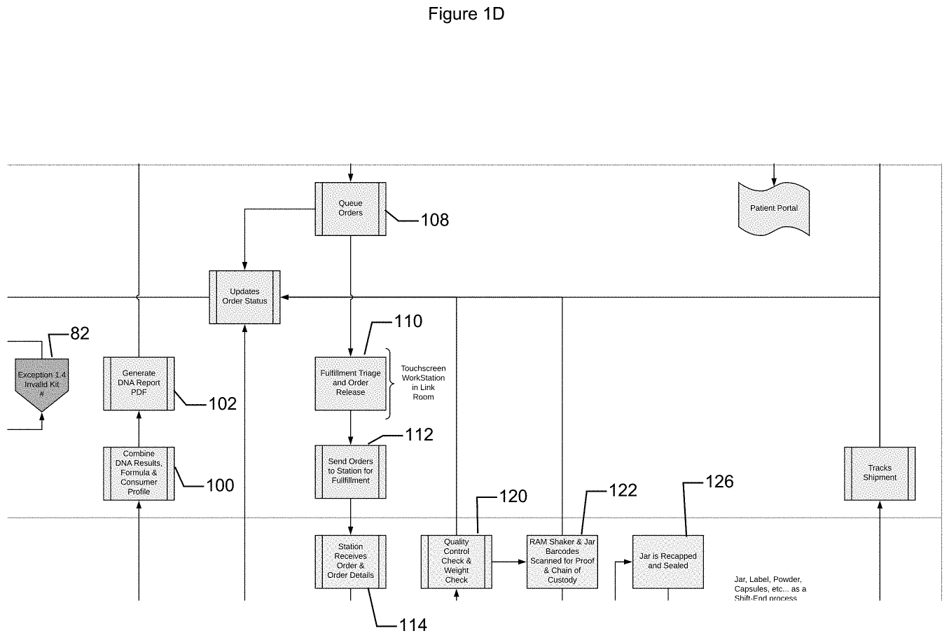

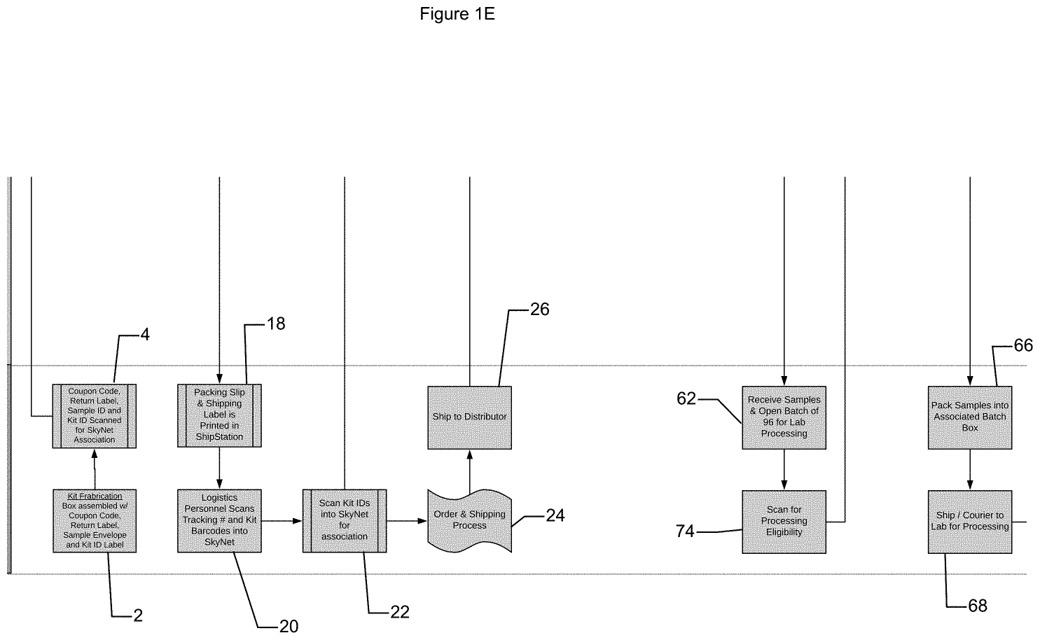

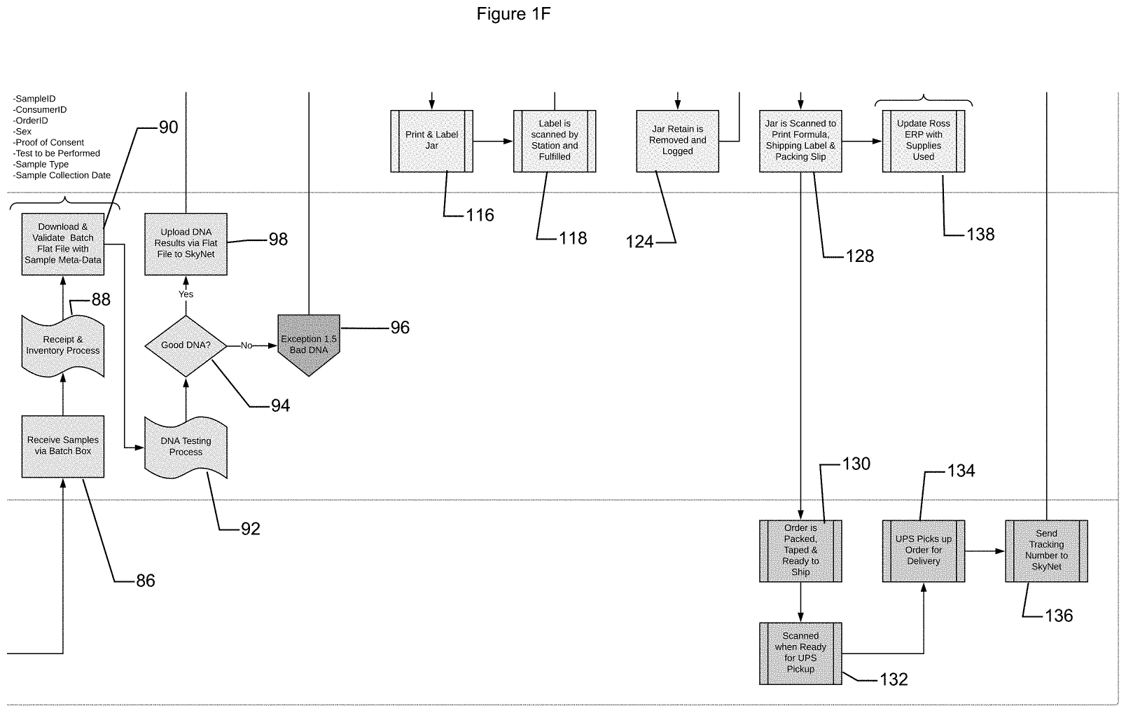

FIG. 1, illustrated in greater detail in FIGS. 1A-1F, illustrates an implementation of an example process performed by one or more portions of the system. Various module(s) of the system may perform one or more operations and/or may communicate with one or more third party providers of information to perform one or more of the operations.

The system may obtain DNA information for a user from pre-existing DNA information and/or from DNA sample(s) obtained from a user. In some implementations, the system may receive DNA information from pre-existing DNA information available to the user. For example, a user may have previously had a DNA analysis (e.g., for health and/or ancestry purposes), and the user may provide and/or provide access to the DNA information from the analysis to the system. The system may communication with one or more third party providers of DNA analysis to receive the DNA information.

In some implementations, the system may receive DNA information from analysis performed on sample(s) provided by users. The system may allow generation of sample kits (e.g., based on order(s) received and/or to generate inventory) (operation 2). For example, the system may transmit a request (e.g., to a shipping module) that one or more sample kits be generated. Sample kits may be generated based on the request from the system. A sample kit may include DNA collection devices (e.g., swab(s), blood collection devices, etc.), sample identification code(s) (e.g., alphanumeric and/or image based code, such as bar codes, QR codes, etc.), coupon code (e.g., associated with ordering nutritional supplements), return packaging (e.g., envelope and/or label), and a kit identification code (e.g., alphanumeric and/or image based code). The sample identification code may be associated with the swabs in the sample kit and/or the kit code may be associated with the sample kit. The kit identification code, sample identification code, and/or information from shipping labels associated with the sample kit may be transmitted to the system for monitoring and/or storage (operation 4).

In some implementations, the sample kit may or may not include other components such as instructions, nutritional supplement information and/or pre-nutrition nutritional supplements. The instructions may include instructions regarding how a user may utilize portions of the sample kit, such as how to utilize swabs, what to do with the swabs upon sample collection, handling of the swab, and/or other appropriate information (e.g., privacy notices, waivers, how to provide user information to the system, etc.). The nutritional supplement information may include information regarding types of nutritional supplements available and/or that may be customized (e.g., flavors, administration forms, etc.) and/or administration instructions. The nutritional supplement information may include information regarding orders for customized nutrition samples and/or other appropriate information. The pre-nutritional nutritional supplement may include one or more compounds to improve and or maintain health of an individual. For example, the pre-nutritional sample may include the second portion of the nutritional formulation that is standardized. The pre-nutrition sample maybe orally administered to the user on an administration schedule (e.g. daily twice daily weekly and or any other appropriate schedule).

In some implementations, the system may associate the sample identification code, the return packaging (e.g., label information for shipping tracking), and/or coupon code with the kit identification code (operation 6). This information or a portion thereof may be stored by a shipping module that manages sample kit information and/or by the system. In some implementations, the shipping module may be inhibited from accessing information received from individuals (e.g., user information). Thus, even if DNA information is determined by a person accessing the sample identification code from the shipping module, this information may not be matched with a specific individual (e.g., to comply with privacy laws and/or increase user satisfaction with the system).

In some implementations, the shipping module may manage inventory based on the sample kits generated (operation 8). For example, the shipping module may track sample kits created, sample kits sent, sample, kits in inventory, component(s) used to generate kits, etc. This information may facilitate ordering components for kits and/or maintaining a predetermined inventory of sample kits for distribution.

Sample kits may be generated to maintain a predetermined inventory and/or upon request (e.g., from the system and/or a user, such as a distributor). A user, such as a distributor, may request one or more sample kits (operation 10). The system may receive the request from the distributor and associate the order for the sample kits with the distributor. For example, a distributor may provide distributor profile information (e.g., user information such as name, address, payment information, etc.) and the system may associate information related to the distributor with the distributor profile (e.g., via a distributor code, etc.).

The system may utilize a marketing module to facilitate sales receipts, order information, and/or other interaction with users such as distributors and/or customers. Thus, the system may receive a request for sample kits from a distributor via the marketing module. The marketing module may manage the sample kit ordering from a distributor (operation 12). The order may be received (operation 14) and a request may be made to the shipping module to allow sample kit order to be fulfilled (operation 16). The shipping module may generate and/or have generated (e.g. via interface with a shipping provider such as UPS, USPS, FedEx, etc.) a shipping label (operation 18). In some implementations, the shipping label may be printed with the packaging information during generation of the sample kits.

The shipping module may receive information related to the shipping label and/or the sample kit order being fulfilled and transmit the information to the system (e.g., for storage and/or monitoring) (operation 20). For example, packaging information, such as a shipping label, for a sample kit may be scanned. Sample kit information may be received by the shipping module and associated with the packaging information and/or the sample kit order being fulfilled (operation 22). For example, a sample kit identification code may be scanned and/or associated with the packaging information. The shipping module may transmit this information and/or the association(s) to the system for storage and/or management. For example, the system may store an association between a distributor profile, sample kit order and information regarding the sample kits (e.g., packaging information and/or sample kit identification code) provided to the distributor in fulfillment and/or partial fulfillment of the order. Thus, the system may be capable of tracking which sample kits are provided to which distributors (operation 24). In some implementations, access to the sample identification code by the distributor may be inhibited. For example, sample kit identification codes may be presented on an external surface of the sample kit while sample identification code(s) may be presented on an internal surface of the sample kit. Thus, a sample kit must be opened for access to the sample identification code(s).

The shipping module may allow the sample kits to be provided to the distributor (e.g., via postal service and/or courier) (operation 24) and/or may update inventory information based on fulfillment of the order. Thus, the order of sample kit(s) may be sent to the distributor (operation 26). The shipping module may transmit confirmation of shipping, transit information, and/or other tracking information regarding the sample kit order to the system.

The system may store information related to the distributor profile and the sample kit(s) provided (operation 28).

The system may transmit to the distributor information related to the order of sample kits, such order status and/or information related to the sample kits. For example, the system may determine the coupon code(s) associated with the sample kits provided to the distributor and transmit the coupon code(s) to the distributor (e.g., via the marketing module) (operation 30). For example, the distributor may be provided with coupon codes to send to customers, marketing information, and/or other information such as invite code(s). The distributor may be provided with information regarding obtaining a DNA sample to facilitate interactions with customers and/or to increase customer satisfaction with the system (e.g., since the distributor can answer questions and/or aid in sample collection).

The distributor may receive the kit (operation 32) and may provide sample kit(s) to customer(s) (operation 34). The system may receive notification (e.g., from the shipping module interacting with the shipping provider) that the distributor has received the kit. In some implementations, the distributor may verify that the sample kit(s) provided are associated with the distributor by verifying sample kit information sent to the user via the marketing module. Although in some implementations, coupon code information may be disposed in the sample kit and may not be accessible by the distributor, the system may transmit to the distributor the coupon code associated with the sample kit (e.g., operation 30).

Customer profile information may be received by the marketing module (operation 36) and/or transmitted to the system (operation 38). For example, a distributor and/or a customer may provide information regarding the customer (e.g., user name, address, credit card, demographic information, etc.) via a marketing module. The customer profile may include information regarding the association of the customer with the distributor and/or the sample kit. For example, the customer may provide the coupon code and/or sample identification code to the system and thus the system may associate the customer profile with this information and/or related information (e.g., sample identification number). The system may generate a customer identification code to associate with the profile, profile information, and/or associations with the customer profile. The system may then request more information from the customer, in some implementations. The system may associate the customer profile with the distributor. For example, the system may transmit the demographic information and/or order information to the marketing module to receive additional information (operation 40).

The system may generate the customer profile (operation 42) in response to receiving the customer profile information. The customer profile may include information such as, but not limited to, name, contact information (e.g., email address, social media account information, phone number, and/or address), identifying information (e.g., demographic information such as birth gender and/or birth date), and/or any other appropriate information (e.g., medications taken, diseases and/or conditions diagnosed, etc.). The system may associate the sample kit identification code with the generated customer profile (operation 44). The sample kit identification code may be provided by the distributor and/or customer to the system and/or determined based on information provided by the user and stored associations (e.g., association between coupon code, invite code and/or sample kit information).

The system may request additional information from the customer. For example, the system may transmit a message (e.g., based on the contact information) to the customer to request the additional information (operation 46). In some implementations, the additional information may not be provided by the distributor (e.g., consent and/or privacy documentation) and/or may be sensitive information that the customer may not want to share with the distributor. By requesting this information separately, user confidence in maintenance of privacy and thus satisfaction with the system may be increased. In some implementations, the message may include a link that directs the customer to a graphical user interface (e.g., generated by the system or portion thereof) to receive the additional information (operation 48). The additional information requested may be based on location, such as state, in which the customer and/or distributor resides (operation 50). For example, compliance with government laws and/or regulations (e.g., state and/or federal) may cause the system to request authorization and/or consent for collection of and/or determination of DNA information and/or customer profile information (operation 52). In some implementations, a determination may be made whether users are requested to provide authorization and/or consent based on the location of the user and/or distributor. In some implementations, users may be requested to provide authorization and/or consent independent of location (e.g., all users may be requested to provide authorization and/or consent). The system may request additional information such as health information and/or consent to DNA testing and/or handling (operation 54).

The customer may before or after providing customer profile information utilize the sample kit. For example, the customer may be administered (e.g., self-administration) pre-nutrition nutritional supplements. As another example, the customer may read information provided in the kit, such as how to obtain samples and/or nutritional information. The customer may obtain a DNA sample using the DNA collection device, such as a swab. For example, a customer may swab a check (e.g., of the customer or of another user) with one or more of the swabs and dispose the swabs in a collection package (e.g., an envelope, a tube within an envelope, etc.). The customer may send the package to allow testing of the DNA on the DNA collection device. For example, the customer may ship the package using a label on the package (e.g., pre-disposed and/or disposed on the package) (operation 56). The shipping module may determine whether the sample has been shipped (58). For example, the shipping module may monitor tracking information associated with a shipping label and/or the shipping module may receive a notification from a shipping provider that a package associated with the shipping label has been received. In some implementations, the system may monitor tracking information for the sample (e.g., to ensure compliance with regulations regarding handling of DNA samples, to provide updates to customers and/or distributors, etc.) (operation 60). The shipping module may transmit notification of shipping and/or at least a portion of the information related to tracking the shipped sample to the system.

Once a determination is made that a sample is received and may be tested, the sample may be transferred to a testing facility. The samples may be transmitted individually and/or in batches. For example, samples from more than one customer may be collected and packaged together in a batch (operation 62). The batch may be assigned a batch identification code. The sample identification code(s) in the batch may be associated with the batch identification codes by the system (operation 64). The samples in the batch may be processed for shipping (operation 66) and transported to the testing facility (operation 68). For example, a shipping label information may be associated with the sample(s), the shipping label information and/or association with the sample(s) may be received by the shipping module, and/or the sample(s) may be transported (e.g., shipped and/or couriered) to a testing facility. The shipping module may transmit the shipping label information, association between sample identification code and/or batch identification code, association between shipping label information and batch and/or sample, and/or shipment tracking information to the system, which may monitor and/or save the received information.

In some implementations, the system may monitor when sample kits are sent to distributors, customer profile information is provided, and/or additional customer information is provided and transmit one or more messages based on this information. For example, if a predetermined amount of time has elapsed from sample kits being sent to a distributor and if customer profile information is not provided to the system, a message may be transmitted to the distributor (e.g., reminder, marketing information, distributor assistance, incentives, etc.). As another example, if a predetermined amount of time has elapsed from the customer profile information being provided and additional customer profile information has not been provided, a message may be transmitted to the distributor and/or customer (e.g., reminder, offering customer assistance, etc.). As another example, if a predetermined amount of time has elapsed from the customer profile information being provided and/or additional customer profile information and the sample shipment information has not been received, a message may be transmitted to the distributor and/or customer (e.g., reminder, offering customer assistance, incentive(s), etc.). For example, the system may verify that a sample has been shipped within approximately 24 hours of providing additional information to the system (operation 70).

As another example, if the sample has not been received after a predetermined amount of time, the system may transmit one or more other messages (operation 72). The system may transmit a message and request confirmation that the customer received the sample kit. If a determination is made that the customer has received the sample kit, one or more reminder messages may be transmitted to the customer and/or distributor. A customer may request a new sample kit. A sample kit may be generated and/or retrieved (e.g., by the shipping module) to be sent to the distributor and/or customer.

A notification may be provided (e.g., by the shipping module) once the sample has been received from the shipping provider and/or a determination may be made whether the sample can be transmitted to the testing facility (operation 74). The customer and/or distributor may be capable of viewing updates to the status sample shipping via the marketing module. If a sample is not received, the customer and/or distributor may be notified and/or a new sample kit may be provided (e.g. to the distributor and/or customer) (operation 76). In some implementations, the system may verify that predetermined information is received prior to testing a received sample. For example, the system may determine whether a customer has provided customer profile information and/or additional information to the system and/or may inhibit testing of the sample if a determination is made that the customer profile information and/or additional information is needed (operation 78). In some implementations, locations may require authorization and/or consent to test samples. The system may determine whether a sample is associated with a customer in which authorization and/or consent is needed and has not been provided (e.g., based on location of the customer and/or distributor) and may inhibit testing of the sample (e.g., until authorization and/or consent is received) (operation 80). The system may transmit messages that include, for example, reminders and/or links to facilitate receipt of information from the customer. In some implementations, a distributor associated with a sample may be determined (e.g., from stored associations) and a message may be transmitted to the distributor based on the authorization and/or consent not received.

In some implementations, the system may inhibit testing of a received sample until predetermined information is verified. For example, a determination may be made whether the sample identification code is valid (e.g., associated with a distributor, unexpired, etc.) and testing of the sample may be inhibited if the sample identification code is not valid (operation 82).

The system may receive a notification that the sample(s) have arrived at a testing facility. For example, the sample(s) (e.g., individually and/or in batch form) may be received at the testing facility (operation 86) and the testing facility may process and/or inventory the sample(s) (operation 88). The system may transmit to the testing facility (e.g., upon a request from the testing facility) a flat file associated with one or more of the samples (operation 90). The flat file may be inhibited from including customer profile information other than sample identification number (e.g., to comply with privacy laws and/or regulations). The flat file may include information related to testing and/or verification of testing. For example, the flat file may include, associated with a sample identification, the type of testing to be performed (e.g., DNA analysis, predetermined SNP mutation testing, gender type, disease markers, etc.), order information, batch information (e.g., identification code), information related to authorization and/or consent (e.g., proof, verification that authorization and/or consent was provided, etc.), birth gender, birth date, sample type, sample collection date, number of associated samples, information about associated samples, and/or any other appropriate information.

The sample(s) provided to the testing facility may be tested (operation 92) and results may be received. In some implementations, a determination may be made whether the sample included DNA appropriate for testing (operation 94). If a determination is made that the sample did not included DNA appropriate for testing (e.g., birth gender did not match DNA sample, sample is contaminated, sample does not include enough DNA, testing is not performable on the sample, etc.), testing may be performed on one or more of the associated samples (e.g., the associated sample may be identified and/or tested) and/or a new sample may be requested from the system (operation 96). The system may allow an associated sample to be delivered to the testing facility if an associated sample is available and/or the system may request shipment of a new sample kit to the distributor associated with the customer and/or the customer. If a determination is made that the sample included DNA appropriate for testing (e.g., testing could be performed, test results matched demographic information such as birth gender, etc.), then the results of the DNA testing requested or a portion thereof may be transmitted to the system (operation 98).

The system may analyze the received DNA testing for sample(s). For example, the system may determine if mutations exist and/or the type of mutations that exist on SNPs of the DNA in a sample. A core formulation may be determined based on the determination of whether mutations exist and/or type(s) of mutations that exist on the SNPs of the DNA in a sample. For example, the system may identify components and/or appropriate amounts of the identified components to be included in the core formulation. The determination of which components to include may be based on research and/or industry studies, in some implementations.

The core formulation may be associated with the consumer profile and/or the DNA test results and/or portions thereof (operation 100). A DNA Report may be generated which may include information such as results of DNA testing, mutations and/or types of mutations in SNPs, and/or the core formulation (operation 102). The core formulation may be transmitted to the system and stored in the system. In some implementations, the DNA Report may be transmitted to and/or presented to the consumer (e.g., via the marketing module). In some implementations, the marketing module may link with (e.g., seamlessly such that the user may not recognize) system to allow the DNA Report to be presented to the user without sharing the information with the marketing module (e.g., to comply with government and/or industry regulations).

Nutritional products based at least partially on the core formulation may be ordered and/or generated by consumer(s). For example, during registration (operation 38) order(s) and/or delivery schedule(s) may be submitted and/or agreed upon (operation 104). Thus, an order may be generated once the core formulation is determined. Order(s) may be requested by user(s) in addition to and/or instead of scheduled delivery (operation 106).

One or more orders (e.g., for the same or different customers) may be received and queue (e.g., based on delivery schedule, order receipt, contents of core formulation, type of nutritional supplement ordered, etc.) (operation 108). In some implementations, the orders may be released to a product generation and/or storage facility (operation 110). For example, the order may be received by and then transmitted from a marketing module to a production module (e.g., capable of controlling the production of for example, core formulation, prenutrition, and/or other appropriate orders). One or more orders may be transmitted to a device capable of generating customized nutritional supplements or portions thereof (operation 112). The device may include stations, such as a station that is capable of generating the nutritional supplement contents and the station(s) may receive information about the order such as pre-nutrition components, customized components (e.g., core formulation), and/or other components. These may form a customized recipe that is fulfilled for an order (operation 114). For example, a container (e.g., a jar) may be obtained and labeled (operation 116). The label may include information based on the order such as customer profile information, core formulation information, nutritional contents of the nutritional supplement, ingredients of the nutritional supplement, administration information, etc. The container may include a container label associated with the order. The container label information may be received by the system (e.g., by scanning the label and transmitting the information to the system) (operation 118). The station may process the container by depositing amounts of core formulation, pre-nutrition, and/or other components (e.g., to form the ordered nutritional component such as tea, shake, flavored drink, etc.) into the container. The container may be shaken for a period of time (e.g., based on core formulation, nutritional supplement type, etc.). A quality control and weight check may be performed (operation 120). For example, the amount of time the container is agitated and/or how the container is agitated may be recorded (e.g., the container label may be scanned before and/or after the agitation). As another example, a weight of the container may be determined and checked against a predetermined expected weight based on the core formulation and/or formulation recipe. In some implementations, the container label and/or equipment labels may be provided to the system (e.g. by scanning labels) to provide chain of custody and/or compliance with processing requirements (e.g., passage through magnetic agitator to identify and/or remove magnetic particles) (operation 122). In some implementations, a portion of the contents of the customized nutritional sample may be removed from the container and separately retained (e.g., to allow testing, recall, and/or compliance with regulations) (operation 124). The container may be closed and/or sealed (operation 126). The seal may facilitate identification of tampering.

The container may then be ready for shipping and delivery to a customer. The container label information and/or shipping label information may be provided to the system (operation 128). This may allow the system to track the container before and/or during delivery. In some implementations, the container may be packaged with other material such as ingredient list, administration schedule, catalog of nutritional supplements, samples etc. The container and/or other items provided may be processed for shipping (operation 130). The packaging label information may be provided to the system (e.g., when picked up by a shipping provider) (operation 132) and the shipping provider may ship the package (operation 134) and provide tracking updates (operation 136).

In some implementations, the system may track the amount of components utilized in the customized nutritional supplement and provide the information to an inventory tracking module (operation 138). Thus, components can be replaced when needed at the station and/or ordered and/or loss due to theft may be inhibited.

In various implementations, order status(es) may be provided to the system, distributor, and/or customer during generation of the customized nutritional supplements.

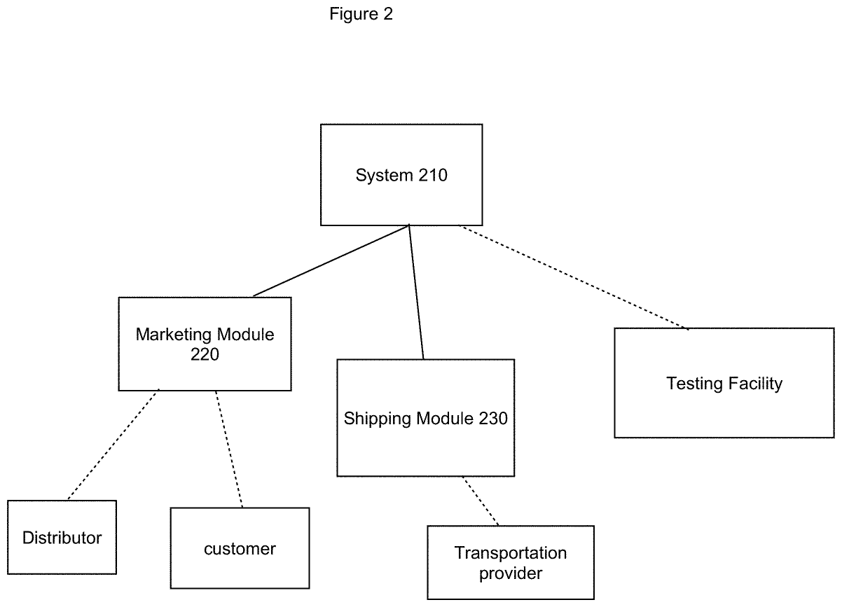

Described processes may be implemented by various systems. In addition, various operations may be added, deleted, and/or modified. In some implementations, a described process may be performed in combination with other processes or portions thereof. For example, a system may be utilized that is similar to the system illustrated in FIG. 2. As illustrated a system 210 may include one or more modules that communicate with the system 210, communication with users such as distributors and/or customers, and/or communicate with third party providers. The communication may be restricted such that the system 210 may be inhibited from transmitting the customer profile information, sample ID, and test results to a module, a distributor, and/or third party provider. Thus, access to sensitive information may be restricted and/or securely stored and utilized by the system. As illustrated, the system 210 may include a marketing module 220 to facilitate communication with users such as distributors and/or customers. The marketing module 220 may generate one or more graphical user interfaces to facilitate request(s) and/or receipt of information from users. The marketing module may allow customer access to secure information, such as test results, stored by the system 210 while restricting sharing of the information with the marketing module. For example, the marketing module may link or otherwise couple with the system 210 such that an interface generated by the system is presented to the user (e.g., via the marketing module). As illustrated, the shipping module 220 may communicate with third parties such as transportation providers (e.g., UPS, USPS, couriers, etc.) and/or one or more testing facilities. The use of the modules separate from the system 210 may facilitate compliance with privacy laws such as HIPAA, which may restrict communications that are allowed within a network.

In various implementations, one or more of the operations may be performed by a third party provider. For example, preparing shipping labels and tracking shipped items may be performed by a third party shipping company. As another example, DNA test results (e.g., from previous testing and/or recent testing) may be obtained from a third party provider. The system may obtain the appropriate authorization from a customer and the DNA test results may be shared by the third party provider with the system. As another example, information may be stored in repositories external to the system (e.g., cloud based repositories, off site repositories, etc.). As another example, the marketing module may be provided by a third party provider that interfaces with the system.

In various implementations, the sample kit may be packaged in a container, such as a shipping package and a label may be generated for the sample kit. The label may not include the sample identification code, in some implementations. Inhibiting presentation of the sample identification code on the shipping container may inhibit unauthorized individuals from accessing sensitive and/or secure information (e.g., test results, customer profile information, etc.).

The sample kit may be sealed in some implementations. The sample kit may include a seal (e.g. tape and or other appropriate seals) that facilitates identification of whether a sample kit has been opened or is on opened. The seal may include adhesive over one or more edges of a container or portions thereof. The seal may be tamper resistant and/or evident. In some implementations, a sell by, use by, and/or expiration date may be provided in association with the sample kit (e.g., swabs and/or pre-nutrition nutritional supplements).

The system may generate and/or store associations between various identification codes, and user profiles such as distributor and/or customer profiles. For example, an association between the sample kit, the sample ID, and the label on the packaging of the sample kit may be determined and/may be stored in a memory of the system. The system may send information regarding the sample kit such as the label information to distributors (e.g., to facilitate tracking and/or record keeping by distributors). The label information may provide an identification code for the distributor to utilize while inhibiting access to the sample identification code (e.g., that may provide access to sensitive and/or secure data).

In various implementations, labels and identification codes may be alpha numeric code(s) and/or images (e.g., bar codes and/or QR codes). A code may be scanned for example to provide the code to the system and/or identify a component (e.g., sample, batch, package, agitator, etc.) to the system. For example, a label code may be scanned by a shipping system and transmitted to the system to be associated with the sample identification code. The codes may facilitate tracking and/or inventory management.

In various implementations, components utilized in the creation of products, such as the sample kit, containers, and/or nutritional supplements may be inventoried. The shipping system may track the inventory associated with the creation of sample kits, for example the system they track the number of swabs in inventory, in prepared sample kids, and/or in sample kids that have been distributed. The shipping system may manage the inventory of sample kids and may request generation/creation of more sample kids based on the inventory of sample kids. The shipping system may track orders received and/or may transmit notifications to have more sample kids created based on the number of orders.

In various implementations, the sample kits may be created on demand (e.g., responsive to a request from a user) and/or may be stocked as inventory.

For example, a batch may include a predetermined number of samples. For example, a testing facility may utilize machines that test a first number of samples. The system may thus, assign a number of samples equal to the first number to a batch and/or assign a number of samples that is a factor of the first number to a batch. For example, 92 samples may be associated with a batch.

In some implementations, when the system receives the authorization and/or consent for the DNA testing, the system may determine whether the sample has been transported (e.g., picked up for transport, in transport, delivered, lost, etc.). The system may transmit a notification to the customer at predetermined time(s) (e.g., periodically, at irregular intervals, etc.) when a determination is made that the sample has not been transported. In some implementations, a system may transmit a notification to the customer and/or distributor if a sample is lost and/or may transmit a new sample kit to allow the customer to retransmit a sample to the user. Since the sample may not include identifying information such as customer profile information, when a sample is lost, compliance with privacy regulations may be maintained.

In some implementations, the DNA test results may include gene mutations on the SNP level for the sample. In some implementations, if a number of gene mutations exceeds a predetermined number, an associated sample may be identified, requested, and tested to determine if the DNA test results were accurate.

In some implementations, the core formulation may be determined by; identifying gene mutations and/or types of gene mutations in the DNA test results and determining which supplements (e.g., type, dosage range, and/or administration schedule) may improve and/or maintain the health of the customer with these identified gene mutations and/or types of gene mutations. The core formulation may thus be a customized formula based on the DNA of a specific user. The core formulation may be utilized in recipes to generate nutritional supplements (e.g., along with pre-nutrition, flavors, stabilizers, additives, etc.).

In some implementations, the core formulations may include one or more cofactors. For example, the system may determine that a genetic mutation on a first SNP causes a first compound to be included in the core formulation. The system may determine whether one or more cofactors should be included based on whether the cofactor is required for (e.g., required for absorption, activation, etc.), increases activation, etc. of the first compound.

In some implementations, the nutritional supplement created and/or the core formulation may be created based on customer dietary preferences. For example, a user may provide dietary preferences related to religion, allergy, lifestyle, diet, and/or goals and the system may store the dietary preferences in the customer profile to allow the core formulation and/or nutritional supplements to be based on the customer preferences. For example, a core formulation may be selected and/or adjusted based on dietary preferences such kosher certification, halal certification, vegan, vegetarian, pescatarian, paleo diet, keto diet, alkaline diet, etc.

In some implementations, the customer may receive the DNA Report, which may include DNA test results, gene mutation information, information associated with the gene mutation (e.g., health and/or symptom information), core formulation, and/or ingredient listing (e.g., type and/or amount) for nutritional supplement ordered. The customer may order one or more nutritional supplements to be customized based on the core formulation and/or dietary preferences. In some implementations, the customer may order one or more non-customized nutritional supplements.

In some implementations, an order may be generated (e.g., automatically and/or manually) when a customer provides the additional information for the customer profile. The order fulfillment may be inhibited until the DNA test results are received such that a core formulation may be utilized, in some implementations. The order may be associated with the coupon code (e.g., that provides a discount, samples, etc. of nutritional supplements). The user may provide authorization for use of payment information provided in the customer profile to pay for the order(s). The system may utilize one or more third party payment systems (e.g., PayPal, bank EFT, etc.) to obtain payment for an order from the customer based on the payment information provided by the user.

In some implementations, the user and/or the system may include default orders. The default orders may include specific nutritional supplements to be delivered and/or a an automatic and/or semi automatic (e.g., confirmatory email transmitted) delivery schedule, in some implementations.

In various implementations, customized nutritional supplement production may be managed by the system and components thereof (e.g., modules of the system that are stored on a memory of the system and perform one or more of the described operations). A first customer identification associated with a sample kit may be generated. An association between a first customer and a first customer identification may be received from a distributor via a marketing module. The association may be stored in a memory. Preliminary customer information may be received via an interface generated by the marketing module. First customer DNA information associated with the first customer may be received from a third party testing system. The customer DNA information may include information relating to SNPs in the DNA of the first customer. The marketing module, production module, and/or the shipping module may be inhibited from requesting access to the received first customer DNA information. One or more first nutritional supplement adjustments may be determined based on the received first customer DNA information. The first nutritional supplemental adjustments or a portion thereof may be received by a production module. The determined one or more first nutritional supplement adjustments may be stored in a memory, in some implementations. The first customer may be allowed to order the customized nutritional supplement based on the one or more first nutritional supplement adjustments via the marketing module by receiving the order from the first customer via the marketing module; determining the first customer identification associated with the first customer from the stored association; and/or retrieving the stored one or more first nutritional supplement adjustments. The customer identification and information related to the one or more first nutritional supplement adjustments may be transmitted to the production module to allow the customized nutritional supplement to be produced. A shipping label based on the first preliminary customer information associated with the first customer may be generated by the shipping module, in some implementations.

Implementations may include one or more of the following features. A determination may be made whether customer DNA information has been received. A notification may be transmitted (e.g., to the third party testing system, to the customer, to the distributor, etc.) if the determination is made that customer DNA information has not been received. The third party testing system may be inhibited from accessing first customer information. The first core formulation may be determined based on the first nutritional supplement adjustments to the production module. The first core formulation based on the first nutritional supplement adjustments may be transmitted to the production module such that the production module may manage the generation of a nutritional supplement based on the first core formulation. A notification may be transmitted to the shipping module to generate a shipping label to be associated with a prenutrition formulation after receiving the association between the first customer identification and the first customer (e.g., in response to receiving an order via the marketing module). A notification may be transmitted to the production module to generate a prenutrition formulation nutritional supplement (e.g., in response to receiving an order via the marketing module). In some implementations, a request for first customer DNA information from a distributor via the marketing module may be received and access to the first customer DNA information may be restricted. A request for first customer DNA information from the first customer may be received via the marketing module, and an interface may be generated within the marketing module that allows presentation of the first customer DNA information. ; Access to the first customer DNA information by the marketing module may be restricted even though the information is presented within an interface of the marketing module, in some implementations. A determination may be made that the preliminary customer information for the first customer has not been received. The distributor that transmitted the association between the customer number and the customer may be determined and a notification may be transmitted to the distributor that the preliminary customer information has not been received. A notification may be received from the third party testing system that the DNA sample provided by the user can not provide the DNA information, and a notification may be transmitted to the shipping module to allow a new DNA sample kit to be sent to the first customer. In some implementations, a DNA Report may be generated based on the received DNA information and the determined one or more nutritional supplement adjustments. In some implementations, dietary preference information may be received from the first customer via the marketing module. Determining at least one of the one or more first nutritional supplement adjustments may include basing the determination on at least partially based on the received dietary preference information. In some implementations, at least one component of the customized nutritional supplement may be adjusted such that the at least one component is removed from the customized nutritional supplement based on the received dietary preference information. A time lapsed since allowing the first customer to order the customized nutritional supplement may be determined. A notification may be transmitted to the marketing module to generate a coupon if a determination is made that the time lapsed is greater than a predetermined maximum time. A time lapsed since a previous order from the first customer may be determined. A notification may be transmitted to the marketing module to generate a coupon if a determination is made that the time lapsed is greater than a predetermined maximum time. The customized nutritional supplement may include a prenutrition set of components and a customized set of components. The first nutritional supplement adjustments may be adjustments made to one or more amounts of one or more components in the customized set of components. In some implementations, additional identification codes associated with the customized nutritional supplement, past orders, the distributor, and/or prenutrition formulation may be received. The system may be able to track customers, orders, and/or distributors based on one or more of the additional identification codes. An ingredient list may be allowed to be generated based on a core formulation and the first nutritional supplement adjustments (e.g., for generation by the system and/or production module of the system to be sent with an order).

In various implementations, the customized nutritional supplement may include a beverage, a food, a powder, a capsule, a tablet, or any other appropriate administration form.

In various implementations, an article may include non-transitory, machine-readable medium storing instructions to perform one or more of the described operations.

In various implementations, a system may perform one or more of the described operations. A memory may store information such as, associations between a plurality of customers and a plurality of customer identifications, customer information for the plurality of customers, nutritional supplement adjustments associated with a plurality of customers, and/or any other appropriate information. One or more processors may perform one or more of the described operations (e.g., separately and/or acting in conjunction with each other). One or more modules may reside on a memory of the system and perform one or more of the operations. For example, a management module may manage the various modules and/or tracking and monitoring within the system. The system may include other modules such as marketing modules, shipping modules, and/or production modules. Access to information available to the management module (e.g., DNA information, customer name associated with DNA information, order history, etc.), may be restricted. For example, marketing, shipping, and/or production modules may be restricted from accessing DNA information and/or customer information associated with DNA information. In various implementations, restricting and managing access to information may facilitate compliance with regulations (e.g., related to DNA and customer profile information, such as HIPPA) and/or increase user satisfaction in the system (e.g., since they know their information is secure).

### EXAMPLES

### Example 1

Sample kits may be sealed kits, in some implementations. A sample kit may include pre-nutrition (e.g., portion 2) in any appropriate form such as powder, drink, capsule, etc.; swabs for DNA sample collection; a coupon code to be associated with a distributor and/or order; and/or information such as a catalog of nutritional supplements, instructions regarding administration schedule for pre-nutrition, and/or instructions for sample collection. The coupon code may be inactive until the system activates the coupon code. The coupon code may be activated by the system when an order is fulfilled using the sample kit associated with the coupon code and/or when a distributor enters the coupon code for activation. The coupon code may be unique to the sample kit.

A sample kit identification code may be associated with the sample kit and may be generated by and/or transmitted to the system. In some implementations, the sample kit identification code may be generated by a shipping module and transmitted to the system.

The shipping module may generate a packing slip and/or transportation label, such as a shipping label identifiable by a third party transportation provider (e.g., USPS). The label may be coupled to the sample kit and/or a packaging of the sample kit. For example, more than one sample kit may be packaged together for order fulfillment for a distributor. As another example, sample kits may be disposed in a package for transportation (e.g., the sample kit may be housed in an envelop for use in returning the sample and/or a fanciful package that may not withstand transportation).

### Example 2

A first user (e.g. a distributor) may request one or more sample kits to be distributed to one or more second users (e.g., customers). A distributor may be a customer, in some implementations. The distributor may utilize a marketing module to request sample kits. The system may associate sample kits provided to fulfill the request (e.g., an order) with the distributor profile information (e.g., distributor identification code, distributor name, distributor contact information, distributor payment information, distributor sales level, etc.).

The distributor may be inhibited from determining the sample identification codes for other users. For example the sample identification code may be on a swab and/or may be disposed in a location inside the packaging of the sample kit. The distributor may be transmitted the sample kit identification code (e.g., as opposed to the sample identification code) and/or a coupon code. The distributor may utilize the sample kit identification code and/or coupon code to track whether customers (e.g., to whom sample kits were distributed) submitted samples and/or ordered nutritional supplements.

In some implementations, the system may receive the request from the distributor and may request generation of a shipping label from a shipping system. The shipping system may print shipping label(s) and/or obtain shipping label(s) for sample kits and may facilitate shipping of the sample kits to the distributor. The system may associate the shipping labels and the sample kits with the distributor that requested the sample kits. The system may associate the shipping labels and the sample identification codes with the distributor. However, in some implementations, the distributor may be inhibited from determining the sample identification code without opening the sample kit. This may increase user satisfaction since sensitive information may be secure in the system.

The distributor may receive the sample kits and provide sample kits to one or more users (e.g., customers). The customers may receive the sample kit and/or may open the sample kit. A distributor may provide a first set of information (e.g. initial registration information for a customer profile such as name and/or contact information) such that a record may be generated and maintained for users to whom the distributor provides a sample kit. The record may be a customer profile. At least a portion of the customer profile may be stored and/or accessible by the marketing module of the system.

The user may utilize swabs within the sample kit. In some implementations, the user may insert the swabs in a cheek, for example, to transfer cells and thus DNA from eh user to the swab. After collection of the sample, the user may insert the swabs into an envelope in the sample kit and/or may seal the swabs in the envelope. The envelope and/or the seal may inhibit deterioration of the DNA samples on the swap for at least 30 days, 3 months, at least 6 months, and/or at least 1 year, in some implementations. The envelope may include shipping information such as a return address, sample kit identification code, and/or customer identification code. In some implementations a sample kit identification code as opposed to a sample identification code maybe identified on a return address, such that the identification of a user (e.g., customer profile information) may not be known outside the system. For example, if a distributor and or other party were to obtain the envelope and/or the swab, by opening the envelope in which the swab was disposed, identifying information of the user (e.g., customer profile information) could not be determined from directly from the code on the envelope.

The user may provide additional information to the system after receiving the sample kit, in some implementations. For example, a graphical user interface of the system (e.g., marketing module) may be provided to the user to receive the additional information. A marketing module that receives the additional information may provide this information to the system. The additional information may include, but is not limited to, birth gender, birth date, other demographic information, health information, address, name, payment information, authorization and/or consent for storage of information and/or testing of sample(s), etc. The user may provide additional information such as a sample kit identification (e.g., which may be disposed on the sample kit, such as on a bottom surface). The system may receive the additional information and store the information (e.g., as a part of a customer profile). In some implementations, the graphical user interface may allow a user to provide an electronic signature in compliance with government and/or industry standards for authorization and/or consent to test and/or store DNA testing information. At least a portion of the customer profile may be stored and/or accessible by the marketing module of the system.

The packaging of the swabs may be transmitted to a location and the return address label may be scanned upon receipt to provide the information to the system.

In some implementations, a distributor may provide the sample kit to a user, but the user may not return the swab(s) of the sample kit. For example, a user may forget, have questions and delay transmission, etc. The system may send one or more reminders to the user and/or distributor related to obtaining a sample kit and not receiving notification of the return of the sample kit.

In some implementations, a distributor may receive one or more notifications based on actions of the user. For example, a distributor may be notified when a sample associated with a sample kit associated with the distributor is or is not shipped. A distributor may be notified when customers order nutritional supplements. A distributor may be notified when customers have questions and request information from the system, in some implementations.

In some implementations, the system may process orders and/or release orders based on the contents of the order, delivery date, and/or any other appropriate information. For example, the contents of an order may be determined and orders or portions thereof with similar nutritional supplements ordered (e.g., type and/or content) may be identified and may be released together for preparation by a device that is capable of generating the customized nutritional supplements. As another example, orders or portions thereof with similar and/or the same labeling (e.g., labeling for an individual, labeling for a product line, etc.) may be identified and released together. Releasing similar orders or portions thereof may facilitate production of the customized nutritional supplements. For example, sending orders or portions thereof with similar labels may minimize label spool changing (e.g., when compared with other types of order processing). As another example, sending orders or portions thereof with similar contents (e.g., in core formulations) may reduce the number of times a storage container is replaced due to different ingredients changing (e.g., when compared with other types of order processing). As another example, sending orders or portions thereof with similar types of nutritional product may facilitate processing (e.g., customized teas may be processed in a batch, customized grape flavored drinks may be processed in a batch), etc.

In some implementations, an ultrasonic agitator may be utilized to agitate customized nutritional supplements that are generated. Using an ultrasonic agitator may increase homogeneity obtained in a shorter time period and/or may decrease noise (e.g., which may cause safety problems).

### End of Examples

In some implementations, the DNA collection device and/sample kit may include a kit for collecting and/or transporting DNA or portions thereof as described in U.S. Pat. No. 6,291,171 entitled “Kit for the non-invasive identification and collection of DNA” filed on Apr. 30, 1999, which is hereby incorporated by reference to the extent it does not conflict with the teachings herein.

In various implementations, the nutritional supplement may be any appropriate form of a nutritional supplement. For example, the nutritional supplement may include capsules, tablets, powders, gels, liquids, etc. In some implementations, the nutritional supplement may be a food or beverage, such as a tea (e.g., the nutritional supplement may include tea and be provided in a form appropriate for brewing). As another example, the nutritional supplement may include food and/or beverages.

In various implementations, a distributer may be a user that for example, provides DNA information, receives a core formulation, and/or receives customized nutritional products.

In various implementations, one or more operations of the system may be performed by one or more modules and/or sub-modules of the system. Although in various implementations operations are described as being performed by a specific module, the operations described may be performed by other modules, sub-modules, and/or the module in conjunction with third party services (e.g., payment systems such as PayPal, shipping services such as USPS, etc.). In some implementations, modules of the system may be restricted from accessing the main system and/or other modules of the system (e.g., to facilitate compliance with privacy laws and/or regulations and/or industry standards). For example, modules may transmit information and/or receive information from the system but may be inhibited from requesting information such as customer profile information, sample identification information and/or associations therewith. For example, the system may store and maintain customer profile information, sample identification information and/or associations therewith but may inhibit other modules of the system from obtaining at least one of these types of information (e.g., one module may have access to customer profile information and not sample information, another module may have access to sample information but not customer profile information, etc.).

In various implementations, the system may utilize an appropriate encryption tool to secure information and/or to comply with government and/or industry standards and/or regulations.

In various implementations, the system may monitor sample kit orders, sample(s), orders of nutritional supplement(s), and/or other operations of the system and may present at least a portion of the monitored information to be presented to distributors and/or customers (e.g., via messages and/or via the marketing module). In some implementations, the system may generate and/or store a chain of custody based on the monitoring of the processing and/or generation of customized nutritional supplements.

In various implementations, retention and/or accessibility of retained samples of customized nutritional supplements may facilitate post processing issues related to ingredient purity, processing contamination, and/or other health concerns. For example, since the system may monitor the custom nutritional supplement generation, items of concern and/or processed proximate (e.g., in location and/or time) may be easily identified.

Although users have been described as a human, a user may be a person, a group of people, a person or persons interacting with one or more computers, and/or a computer system. Various operations may be performed automatically, semi-automatically, and/or manually. For example, a container may be transported to an agitator manually and/or automatically.

One or more of the described operations may be performed by data processing apparatus, where an article that includes a machine-readable medium stores instructions operable to cause the data processing apparatus to perform the described operations.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

In various implementations, module(s) of the system, such as the management module, the shipping module, a production module, etc. may perform one or more of the operations as described in FIGS. 1A-F.

One or more of the processes illustrated in FIGS. 1A-F or portions thereof may be implemented by various systems, such as the systems described in FIG. 2. In addition, various operations of FIGS. 1A-F or portions thereof may be added, deleted, and/or modified.

These computer programs (also known as programs, software, software applications or code) include machine instructions for a programmable processor, and can be implemented in a high-level procedural and/or object-oriented programming language, and/or in assembly/machine language. As used herein, the term “machine-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device (e.g., displays such as LED, LCD; touch screen; etc) for displaying information to the user and an input device (e.g., a touch screen, a keyboard, a pointing device such as a mouse or a track pad) by which the user can provide input to the computer. Other kinds of devices can be used to provide for interaction with a user as well; for example, feedback provided to the user by an output device can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), and the Internet.

The computing system may include clients and servers. A client and server are generally remote from each other and typically interact through a communication network. The relationship of client and server arises by virtue of computer programs running on the respective computers and having a client-server relationship to each other. Updates to the system may be pushed to clients via a network.

One or more graphical user interface (GUI) of the interface(s) generated by the system may be displayed on a presentation interface of the user device (e.g., user device of the customer, distributor, users of modules such as shipping and/or production modules running on clients, etc.), such as a monitor or screen, of the client. GUI may be operable to allow the user of a user device to interact with the system or portions thereof. Generally, GUI provides a user with an efficient and user-friendly presentation of data provided by the System. GUI includes a plurality of displays having interactive fields, such as icons, tabs, pull-down lists, fillable fields, and editable text operated by the user. And in one example, GUI presents an explore-type interface and receives commands from the user. It should be understood that the term graphical user interface may be used in the singular or in the plural to describe one or more graphical user interfaces in each of the displays of a particular graphical user interface. Further, GUI contemplates any graphical user interface, such as a generic web browser, that processes information in the system and/or user device and efficiently presents the information to the user. In some implementations, GUI may present a web page embedding content. The server can accept data from a user device(s) via the web browser (e.g., Microsoft Internet Explorer, Safari, or Google Chrome) and return the appropriate Hyper Text Markup Language (HTML) or eXtensible Markup Language (XML) responses.

Although FIG. 2 provides one example of an system (e.g., server(s)) that may be used with the disclosure, the server can be implemented using computers other than servers, as well as a server pool. For example, a server may include a general-purpose personal computer (PC), a Macintosh, a workstation, a UNIX-based computer, a server computer, or any other suitable device. According to one implementation, a server may include a web server. Server may be adapted to execute any operating system including UNIX, Linux, Windows, or any other suitable operating system. In short, server may include software and/or hardware in any combination suitable to provide access to data and/or translate data to an appropriate compatible format.

Although a single processor has been described in the sysetm and/or user devices, multiple processors may be used according to particular needs, and reference to processor is meant to include multiple processors where appropriate. Processor may include a programmable logic device, a microprocessor, or any other appropriate device for manipulating information in a logical manner.

A memory of the system, memory of user device(s), and/or additional repositories may be any appropriate form of memory. For example, additional repositories may include a relational database. However, a variety of repositories may be used, such as, SQL databases, relational databases, object oriented databases, distributed databases, XML databases, and/or web server repositories. Furthermore, memory may include one or more forms of memory such as volatile memory (e.g., RAM) or nonvolatile memory, such as read-only memory (ROM), optical memory (e.g., CD, DVD, or LD), magnetic memory (e.g., hard disk drives, floppy disk drives), NAND flash memory, NOR flash memory, electrically-erasable, programmable read-only memory (EEPROM), Ferroelectric random-access memory (FeRAM), magnetoresistive random-access memory (MRAM), non-volatile random-access memory (NVRAM), non-volatile static random-access memory (nvSRAM), and/or phase-change memory (PRAM).

Although FIG. 2 illustrates an implementation that may be utilized with the system, other appropriate systems may be utilized.

It is to be understood the implementations are not limited to particular systems or processes described which may, of course, vary. It is also to be understood that the terminology used herein is for the purpose of describing particular implementations only and is not intended to be limiting. As used in this specification, the singular forms “a”, “an” and “the” include plural referents unless the content clearly indicates otherwise. Thus, for example, reference to “a compound” includes a combination of two or more compounds and reference to “a supplement” includes different types and/or combinations of supplements.

Although the present disclosure has been described in detail, it should be understood that various changes, substitutions and alterations may be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A method of managing customized nutritional supplement production, the method comprising:
generating a first customer identification associated with a sample kit;
receiving an association between a first customer and a first customer identification from a distributor via a marketing module and storing the association in a memory;
receiving preliminary customer information via an interface generated by the marketing module;
receiving first customer DNA information associated with the first customer from a third party testing system, wherein the customer DNA information comprises information relating to SNPs in the DNA of the first customer, wherein the marketing module is inhibited from requesting access to the received first customer DNA information;
determining one or more first nutritional supplement adjustments based on the received first customer DNA information, wherein the one or more first nutritional supplemental adjustments or a portion thereof are received by a production module;
storing the determined one or more first nutritional supplement adjustments in a memory;
allowing the first customer to order the customized nutritional supplement based on the one or more first nutritional supplement adjustments via the marketing module by:
receiving the order from the first customer via the marketing module;
determining the first customer identification associated with the first customer from the stored association;
retrieving the stored one or more first nutritional supplement adjustments;
transmitting the customer identification and information related to the one or more first nutritional supplement adjustments to the production module to allow the customized nutritional supplement to be produced; and
generating a shipping label based on the first preliminary customer information associated with the first customer.

2. The method of claim 1 further comprising:
determine whether customer DNA information has been received;
transmitting a notification if the determination is made that customer DNA information has not been received

3. The method of claim 1 wherein the third party testing system is inhibited from accessing first customer information.

4. The method of claim 1 further comprising:
determining a first core formulation based on the first nutritional supplement adjustments to the production module; and
transmitting the first core formulation based on the first nutritional supplement adjustments to the production module such that the production module may manage the generation of a nutritional supplement based on the first core formulation.

5. The method of claim 1 further comprising:
transmitting a notification to the shipping module to generate a shipping label to be associated with a prenutrition formulation after receiving the association between the first customer identification and the first customer; and
transmitting a notification to the production module to generate a prenutrition formulation nutritional supplement.

6. The method of claim 1, further comprising:
receiving a request for first customer DNA information from a distributor via the marketing module; and
restricting access to the first customer DNA information.

7. The method of claim 1 further comprising:
receiving a request for first customer DNA information from the first customer via the marketing module; and
generating an interface within the marketing module that allows presentation of the first customer DNA information; and
restricting access to the first customer DNA information by the marketing module.

8. The method of claim 1 further comprising:
determining that the preliminary customer information for the first customer has not been received;
determining the distributor that transmitted the association between the customer number and the customer;
transmitting a notification to the distributor that the preliminary customer information has not been received.

9. The method of claim 1 further comprising:
receiving a notification from the third party testing system that the DNA sample provided by the user can not provide the DNA information;
transmitting a notification to the shipping module to allow a new DNA sample kit to be sent to the first customer.

10. The method of claim 1 further comprising:
generating a DNA Report based on the received DNA information and the determined one or more nutritional supplement adjustments.

11. The method of claim 1 further comprising:
receiving dietary preference information from the first customer via the marketing module;
wherein determining at least one of the one or more first nutritional supplement adjustments further comprises determining one or more first nutritional supplement adjustments based on the received dietary preference information.

12. The method of claim 11 further comprising
adjusting at least one component of the customized nutritional supplement such that the at least one component is removed from the customized nutritional supplement based on the received dietary preference information.

13. The method of claim 1 further comprising:
determining a time lapsed since allowing the first customer to order the customized nutritional supplement;
transmitting a notification to the marketing module to generate a coupon if a determination is made that the time lapsed is greater than a predetermined maximum time.

14. The method of claim 1 further comprising:
determining a time lapsed since a previous order from the first customer;
transmitting a notification to the marketing module to generate a coupon if a determination is made that the time lapsed is greater than a predetermined maximum time.

15. The method of claim 1 wherein the customized nutritional supplement comprises a prenutrition set of components and a customized set of components, and wherein the first nutritional supplement adjustments are adjustments made to one or more amounts of one or more components in the customized set of components.

16. The method of claim 1 further comprising:
receiving one or more additional identification codes associated with at least one of the customized nutritional supplement, past orders, the distributor, or prenutrition formulation; and
allow tracking based on one or more of the additional identification codes.

17. The method of claim 1 further comprising:
transmitting an ingredient list based on a core formulation and the first nutritional supplement adjustments for generation by the production module to be sent with an order.

18. The method of claim 1 wherein the customized nutritional supplement comprises at least one of a beverage, a food, a powder, a capsule, or a tablet.

19. An article comprising non-transitory, machine-readable medium storing instructions to perform one or more operations, wherein the one or more operations are adapted to:
generating a first customer identification associated with a sample kit;
receiving an association between a first customer and a first customer identification from a distributor via a marketing module and storing the association in a memory;
receiving preliminary customer information via an interface generated by the marketing module;
receiving first customer DNA information associated with the first customer from a third party testing system, wherein the customer DNA information comprises information relating to SNPs in the DNA of the first customer, wherein the marketing module is inhibited from requesting access to the received first customer DNA information;
determining one or more first nutritional supplement adjustments based on the received first customer DNA information, wherein the one or more first nutritional supplemental adjustments or a portion thereof are received by a production module;
storing the determined one or more first nutritional supplement adjustments in a memory;
allowing the first customer to order the customized nutritional supplement based on the one or more first nutritional supplement adjustments via the marketing module by:
receiving the order from the first customer via the marketing module;
determining the first customer identification associated with the first customer from the stored association;
retrieving the stored one or more first nutritional supplement adjustments;
transmitting the customer identification and information related to the one or more first nutritional supplement adjustments to the production module to allow the customized nutritional supplement to be produced; and
generating a shipping label based on the first preliminary customer information associated with the first customer.

20. A system comprising:
a memory storing:
associations between a plurality of customers and a plurality of customer identifications;
customer information for the plurality of customers;
nutritional supplement adjustments associated with a plurality of customers; and
one or more processors configured to execute one or more instructions to:
generate a first customer identification associated with a sample kit;
receive an association between a first customer and a first customer identification from a distributor via a marketing module and storing the association in a memory;
receive preliminary customer information via an interface generated by the marketing module;
receive first customer DNA information associated with the first customer from a third party testing system, wherein the customer DNA information comprises information relating to SNPs in the DNA of the first customer, wherein the marketing module is inhibited from requesting access to the received first customer DNA information;
determine one or more first nutritional supplement adjustments based on the received first customer DNA information, wherein the one or more first nutritional supplemental adjustments or a portion thereof are received by a production module;
store the determined one or more first nutritional supplement adjustments in a memory;
allow the first customer to order the customized nutritional supplement based on the one or more first nutritional supplement adjustments via the marketing module by:
receiving the order from the first customer via the marketing module;
determining the first customer identification associated with the first customer from the stored association;
retrieving the stored one or more first nutritional supplement adjustments;
transmitting the customer identification and information related to the one or more first nutritional supplement adjustments to the production module to allow the customized nutritional supplement to be produced; and
generating a shipping label based on the first preliminary customer information associated with the first customer.
